SEQUENCE LISTING (22 sequences)

SEQ ID NO: 1 - protein, 100 aa, Artificial sequence
  VARIANT (1)...(100): MB-1Trp
MATEDMTDMMTTLFKTMQLLTKSEPTAMDEATKTATTMKNHLQNLMQKTKNKEDMTDMATTWFKTMQLLTKTEPSAMDEATKTATTMKNHLQNLMQKGVA

SEQ ID NO: 2 - protein, 100 aa, Artificial sequence
  VARIANT (1)...(100): MB-1TrpRH
MATEDMTDMCTTLFKTMQLLTKSEPTAMDEATKTATTMKNHLQNLMQKTKNKEDMTDMATTWFKTMQLLTKTEPSAMDEATKTATTMKNHCQNLMQKGVA

SEQ ID NO: 3 - protein, 100 aa, Artificial sequence
  VARIANT (1)...(100): MB-1LH
MATEDMTDMMTTCFKTMQLLTKSEPTAMDEATKTATTMKNHLQNLMQKTKNKEDMTDMATTYFKTMQLLTKTEPSAMDEATKTATTCKNHLQNLMQKGVA

SEQ ID NO: 4 - protein, 100 aa, Artificial sequence
  VARIANT (1)...(100): Recombinant molecules
MATEDMTDMCTTLFKTMQLLTKSEPTAMDEATKTATTMKNHLQNLMQKTKNKEDMTDMATTYFKTMQLLTKTEPSAMDEATKTATTMKNHCQNLMQKGVA

SEQ ID NO: 5 - protein, 100 aa, Artificial sequence
  VARIANT (1)...(100): MB-1TrpLH
MATEDMTDMMTTCFKTMQLLTKSEPTAMDEATKTATTMKNHLQNLMQKTKNKEDMTDMATTWFKTMQLLTKTEPSAMDEATKTATTCKNHLQNLMQKGVA

SEQ ID NO: 6 - DNA, 303 bp, Artificial sequence
  gene (1)...(303): MB-1Trp
atggctacggaagacatgaccgacatgatgaccaccctgtttaaaactatgcagctgttgaccaagtcggaacccacggctatggacgaggccactaaaacggctactacaatgaagaatcatcttcaaaacctgatgcagaagactaagaacaaagaagacatgacggacatggccactacgtggttcaaaacgatgcagttgttaacgaagaccgacccctcggccatggacgaggccacgaagacggctacaaccatgaaaaatcatctgcagaacttgatgcaaaaaggcgtagcttaa

SEQ ID NO: 7 - DNA, 302 bp, Artificial sequence
  gene (1)...(302): MB-1TrpRH
atggctacggaagacatgaccgacatgtgcaccaccctgtttaaaactatgcagctgttgaccaagtcggaacccacggctatggacgaggccactaaaacggctactacaatgaagaatcatcttcaaaacctgatgcagaagactaagaacaaagaagacatgacggacatggccactacgtgcttcaaaacgatgcagttgttaacgaagaccgagccctcggccatggacgaggccacgaagacggctacaaccatgaaaaatcattgccagaacttgatgcaaaaaggcgtagctta

SEQ ID NO: 8 - DNA, 303 bp, Artificial sequence
  gene (1)...(303): MB-1LH
atggctacggaagacatgaccgacatgatgaccacctgctttaaaactatgcagctgttgaccaagtcggaacccacggctatggacgaggccactaaaacggctactacaatgaagaatcatcttcaaaacctgatgcagaagactaagaacaaagaagacatgacggacatggccactacgtacttcaaaacgatgcagttgttaacgaagaccgagccctcggccatggacgaggccacgaagacggctacaacctgcaaaaatcatctgcagaacttgatgcaaaaaggcgtagcttaa

SEQ ID NO: 9 - DNA, 303 bp, Artificial sequence
  gene (1)...(303): MB-1RH
atggctacggaagacatgaccgacatgtgcaccaccctgtttaaaactatgcagctgttgaccaagtcggaacccacggctatggacgaggccactaaaacggctactacaatgaagaatcatcttcaaaacctgatgcagaagactaagaacaaagaagacatgacggacatggccactacgtacttcaaaacgatgcagttgttaacgaagaccgagccctcggccatggacgaggccacgaagacggctacaaccatgaaaaatcattgccagaacttgatgcaaaaaggcgtagcttaa

SEQ ID NO: 10 - DNA, 303 bp, Artificial sequence
  gene (1)...(303): MB1-TrpLH
atggctacggaagacatgaccgacatgatgaccacctgctttaaaactatgcagctgttgaccaagtcggaacccacggctatggacgaggccactaaaacggctactacaatgaagaatcatcttcaaaacctgatgcagaagactaagaacaaagaagacatgacggacatggccactacgtggttcaaaacgatgcagttgttaacgaagaccgagccctcggccatggacgaggccacgaagacggctacaacctgcaaaaatcatctgcagaacttgatgcaaaaaggcgtagcttaa

SEQ ID NO: 11 - DNA, 27 bp, Artificial sequence
  primer_bind (1)...(27): Primer MB-1
atggccactacgtacttcaaaacgatg

SEQ ID NO: 12 - DNA, 27 bp, Artificial sequence
  primer_bind (1)...(27): Primer Tyr62Trp
atggccactacgtggttcaaaacgatg

SEQ ID NO: 13 - DNA, 27 bp, Artificial sequence
  primer_bind (1)...(27): Primer MB-1
atgatgaccaccctgtttaaaactatg

SEQ ID NO: 14 - DNA, 27 bp, Artificial sequence
  primer_bind (1)...(27): Primer L13C
atgatgaccacctgctttaaaactatg

SEQ ID NO: 15 - DNA, 27 bp, Artificial sequence
  protein_bind (1)...(27): Primer MB-1
acggctacaaccatgaaaaatcatctg

SEQ ID NO: 16 - DNA, 27 bp, Artificial sequence
  protein_bind (1)...(27): Primer M87C
acggctacaacctgcaaaaatcatctg

SEQ ID NO: 17 - DNA, 27 bp, Artificial sequence
  protein_bind (1)...(27): Primer MB-1
atgaccgacatgatgaccaccctgttt

SEQ ID NO: 18 - DNA, 27 bp, Artificial sequence
  primer_bind (1)...(7): Primer M10C
atgaccgacatgtgtaccaccctgttt

SEQ ID NO: 19 - DNA, 26 bp, Artificial sequence
  primer_bind (1)...(26): Primer MB-1
atgaaaaatcatctgcagaacttgat

SEQ ID NO: 20 - DNA, 27 bp, Artificial sequence
  primer_bind (1)...(27): Primer L91C
atgaaaaatcattgccagaacttgatg

SEQ ID NO: 21 - DNA, 24 bp, Artificial sequence
  primer_bind (1)...(24): MB-1
atggccactacgtacttcaaaacg

SEQ ID NO: 22 - DNA, 24 bp, Artificial sequence
  primer_bind (1)...(24): Primer Y62W
atggccactacgtggttcaaaacg

(19) United States
(12) Patent Application Publication
(10) Pub. No.: US 2003/0118573 A1
(43) Pub. Date: Jun. 26, 2003

(54) MB-1 analogs and uses thereof

(75) Inventors: Marc Beauregard, Cap-Sante (CA); Mylene-Claude Gagnon, Cap-Sante (CA); Alain Doucet, L'Ancienne-Lorette (CA); Martin Williams, Quebec (CA)

Correspondence Address: OGILVY RENAULT, 1981 MCGILL COLLEGE AVENUE, SUITE 1600, MONTREAL, QC, H3A2Y3, CA

(73) Assignee: AGROTERRA BIOTECH INC., Trois-Rivieres (CA)

(21) Appl. No.: 10/272,929

(22) Filed: Oct. 18, 2002

Related U.S. Application Data
(60) Provisional application No. 60/329,759, filed on Oct. 18, 2001.

(51) Int. Cl. A61K 48/00; A61K 38/43

(52) U.S. Cl. 424/94.1; 514/44

(57) ABSTRACT

The present invention relates to protein engineering for production of analogs of MB-1. MB-1 analogs are more stable to different biochemical conditions than the native protein, and allow for improving the supplying of essential amino acids to human and animals. De novo synthesis of MB-1 analogs is principally targeted to enhance availability of essential amino acids required for farm animal production or human health.

### BACKGROUND OF THE INVENTION

1. Field of the Invention

The invention relates to protein design or protein engineering in the production of polypeptides allowing delivery of food essential amino acids. This invention particularly relates to de novo synthesis of polypeptide designed to enhance availability of amino acids required for milk production in cows, farm animal production or human food.

2. Description of Prior Art

Diet is important in determining general health, work performance, energy level and appearance. An equilibrated diet should include proper amounts of essential nutrients, which are the nutritive elements that an animal is incapable of synthesizing by itself and therefore, that must be obtained from food. As regimen is highly variable throughout animal kingdom, essential nutrient requirements are significantly different for each animal specie. In humans, approximately forty essential nutrients are required, wherein fourteen are minerals, fourteen are vitamins, ten are amino acids and two are essential fatty acids.

Other non-essential, but beneficial, elements can also be obtained from food, including enzymes, beneficial bowel bacteria and certain kinds of fiber. Ultimately, supplying these factors can become essential, especially when the diet contains highly processed foods. Indeed, extensively processed foods fail to provide all the nutritional elements required for optimum animal or human health since processing destroys the chemically fragile essential nutritional elements. For example, processing steps may expose the carbon-carbon double bonds in the hydrocarbon chains of essential fatty acids to oxidizing agents such as light and oxygen, which cause the lost of their nutritional value.

Despite their publicized deficiencies, processed aliments have become popular due to the ease, speed and convenience with which meals can be prepared. As a result of a diet rich in such aliments, humans and animals often suffer from a nutritional deficiencies. This reality poses a particular problem in animal feeding strategies since many of the commonly available blends of feed tend to be somewhat deficient in certain key amino acids. As a result, the production of amino acid supplements for enhancing animal feed use has become an important worldwide business.

The amino acid supplements can be produced either by fermentation or by chemical means. These pure amino acids or amino acid analogs are then added at the required levels to provide a balanced protein diet for feeding the animal. As example, the amino acid methionine, the aqueous solutions of methionine salts, in particular sodium methionine, and substitutes such as the methionine hydroxy analogue (MHA) are used all over the world as feed additives for breeding poultry, pigs and other economically useful animals, and to stimulate the production of animal protein. In addition to its use in meat production purpose, methionine play a role in milk production, being a limiting amino acid for milk protein production. A well balanced level of methionine is therefore thought to result in effective levels of milk protein production and in an increase in milk production.

To benefit from these advantages, methionine can be added directly to animals food. However, the native form of methionine is rapidly degraded by bacteria in the rumen of bovine and, consequently, only a small portion of the methionine enters the bloodstream. To overcome this problem, a general strategy consisting in introducing methionine into the diet in a protected or modified form, allows the compound to pass through the rumen and to remain unaffected. The methionine is then released from the protected or modified form and passes trough the small intestine, being further absorbed into the bloodstream.

One of the most widely studied compounds for this particular purpose is the hydroxy analogue of methionine 2-hydroxy-4-(methylthio) butanoic acid. International Patent application published under number WO 99/04647 discloses a method for introducing methionine into the rumen by supplementing the feed with an hydroxy analogue of methionine. In this patent application, it is claimed that the hydroxy analogue is substantially unaffected by rumen degradation, passing through the rumen and consequently providing at least 20%, preferably at least 40% of the hydroxy analogue for absorption into the bloodstream through the intestine. The patent application refers to the hydroxy analogue, its salts, esters, amides and oligomers as being “rumen by-pass” and claims an improved efficient means of introducing methionine into the bloodstream of the cow. It is disclosed that the compounds by-pass the rumen and are absorbed in the intestine.

Although production of methionine as food additive is currently affordable, some amino acids are still too expensive or difficult to handle to be widely used at this time. The expense associated with feed additives used for animal production led to the study of intracellular production of high-quality protein by transgenic crops and other organisms, as mean for obtaining efficient and low cost sources of essential amino acids (EAA).

Three main approaches for improving protein quality in a given organism have been explored. The first approach involves transferring a gene coding for a high-quality protein from one organism to another that is more suitable for farming practices (heterologous expression). Recently, soybean and sunflower albumins have been chosen for their high methionine contents for the development of transgenic crops by major agro-biotechnology companies. With this approach, the amino acid composition of a natural protein is naturally predetermined and may not be conformed to the desired EAA ratio. The second approach involves modifying the genes of an organism so that specified EAAs are incorporated into the proteins. This strategy, however, often destabilize the protein and/or prevent it from folding, which may jeopardize its recovery.

U.S. Pat. No. 5,939,599 describes methionine enriched plants that have been engineered.

U.S. Pat. No. 6,169,232 describes tryptophan enriched plants that have been genetically engineered. Despite the result achieved, it remains that previous protein engineering projects focused on a single EAA at a time, in the context of a natural protein with a given composition.

The last approach involves creating a new protein with a biased composition of selected EAAs, an approach commonly called de novo design. Theoretically, this strategy allows for full control of the amino acid composition of the protein and is, thus, an advantage over the previously mentioned options. De novo design of artificial proteins is an emerging area of research that rely on the understanding of protein structure to allow the creation of molecules with desirable and specific structures and properties. De novo protein design received considerable attention in the last years, allowing significant advances to be made in the attempt to reach the goal of producing stable, well-folded proteins with novel sequences.

Efforts to design proteins are based on knowledge of the physical properties that determine protein structure, such as the patterns of hydrophobic and hydrophilic residues in the sequence, salt bridges bonds, hydrogen bonds, and secondary structural preferences of amino acids.

Various approaches to apply these principles have been attempted. For example, the construction of alpha-helical and beta-sheet proteins with native-like sequences was attempted by individually selecting the residue required at every position in the target fold. Alternatively, a minimalist approach was used to design helical proteins, where the simplest possible sequence believed to be consistent with the folded structure was generated, with varying degrees of success. An experimental method that relies on the hydrophobic and polar (HP) pattern of a sequence was developed where a library of sequences with the correct pattern for a four helix bundle was generated by random mutagenesis. Among non de novo approaches, domains of naturally occurring proteins have been modified or coupled together to achieve a desired tertiary or quaternary organization.

Though the correct secondary structure and overall tertiary organization seem to have been attained by several of the above techniques, many designed proteins appear to lack the structural specificity of native proteins. The complementary geometric arrangement of amino acids in the folded protein is the root of this specificity and is encoded in the sequence.

Recent studies using coiled coils have demonstrated that core side-chain packing can be combined with explicit backbone flexibility. In these cases, the goal was to search for backbone coordinates that satisfied a fixed amino acid sequence. Recently, an algorithm for precise engineering of protein cores was reported.

It would be highly desirable to be provided with new engineered polypeptides capable of acting as amino acid delivery system. It would be desirable also to be provided with new engineered protein that would be a source of selected EAA in given proportions, and be sufficiently stable as to resist degradation in a production host (bacteria or plant).

### SUMMARY OF THE INVENTION

One object of the present invention is to provide a method for improving the supplying of at least one essential amino acid to a human or an animal, which comprises administering to a human or an animal at least one polypeptide having an amino acid sequence selected from the group consisting of SEQ ID NO: 1, SEQ ID NO: 2, SEQ ID NO: 3, SEQ ID NO: 4, and SEQ ID NO: 5, or a fragment thereof between 10 to 90 amino acids, or an encoding nucleic acid thereof, which may be a DNA or a RNA.

The polypeptide can be administered orally to a human or an animal.

The essential amino acid may be a methionine, lysine, threonine, leucine, tryptophan, an analog or a derivative thereof. The animal may be selected from a mammal, a bird, or a fish.

Another object of the present invention is to provide a composition comprising at least one polypeptide having an amino acid sequence selected from the group consisting of SEQ ID NO: 1, SEQ ID NO: 2, SEQ ID NO: 3, SEQ ID NO: 4, and SEQ ID NO: 5 for endowing a human or an animal with at least one essential amino acid by oral administration.

One object of the invention is to provide a nucleotide sequence selected from the group consisting of SEQ ID NO: 6, SEQ ID NO: 7, SEQ ID NO: 8, SEQ ID NO: 9 and SEQ ID NO: 10, or any other nucleic acid sequence under form of a DNA or a RNA, coding for at least one polypeptide consisting of an amino acid sequence selected from the group consisting of SEQ ID NO: 1, SEQ ID NO: 2, SEQ ID NO: 3, SEQ ID NO: 4 and SEQ ID NO: 5, or an analog or fragment thereof.

Also, in accordance with the present invention there is provided an expression vector comprising a nucleotide sequence selected from the group consisting of SEQ ID NO: 6, SEQ ID NO: 7, SEQ ID NO: 8, SEQ ID NO: 9 and SEQ ID NO: 10, designed for the production of a recombinant polypeptide consisting of the amino acid sequence as set forth in SEQ ID NO: 1, SEQ ID NO: 2, SEQ ID NO: 3, SEQ ID NO: 4, and SEQ ID NO: 5, the recombinant peptide being produced in a bacteria, yeast or an eukaryotic cell.

Another object of the invention is to provide a cell transformed with the expression vector comprising a nucleotide sequence selected form the group consisting of SEQ ID NO: 6, SEQ ID NO: 7, SEQ ID NO: 8, SEQ ID NO: 9 and SEQ ID NO: 10.

### DESCRIPTION OF THE DRAWINGS

FIG. 1 illustrates MB-1's hydrophobic core as predicted by design;

FIG. 2 illustrates a circular dichroism spectra of MB-1Trp;

FIG. 3 illustrates the unfolding of MB-1Trp as monitored by fluorospectroscopy;

FIG. 4 illustrates the enhancement of ANSA fluorescence by MB-1Trp;

FIG. 5 illustrates a thermal denaturation curve for MB-1Trp;

FIG. 6 illustrates the degradation of MB1 and MB1 Trp mutant using Pronase E;

FIG. 7 illustrates a schematic representation of MB-1LH and MB-1RH;

FIG. 8 illustrates CD spectra of the four mutants;

FIG. 9 illustrates CD spectra measured in the presence of DTT;

FIG. 10 illustrates examples of thermal denaturation curves;

FIG. 11 illustrates the proteolytic degradation of the proteins;

FIG. 12 illustrates an SDS-PAGE analysis of MB-1TrpRH;

FIG. 13 illustrates a circular dichroism spectra of MB-1 mutants;

FIG. 14 illustrates thermal denaturation curves for MB-1RH and MB-1Trp.

FIG. 15 illustrates a design of MB-1RH; and

FIG. 16 illustrates the degradation of MB-1(WT) and mutants by pronase E.

### DETAILED DESCRIPTION OF THE PREFERRED EMBODIMENT

In accordance with the present invention, there is provided a method for supplying at least one essential amino acid (EAA) to a human or an animal, by administering to the human or the animal at least one polypeptide having an amino acid sequence selected from the group consisting of SEQ ID NO: 1, SEQ ID NO: 2, SEQ ID NO: 3, SEQ ID NO: 4, and SEQ ID NO: 5. The polypeptides of the invention may be delivered to a human or an organisms by oral, intravenous, intraperitoneal, or percutaneous administration. Although a full length polypeptide is preferred, functional fragments are also included in the present invention. Functional fragment comprises between 10 and 90 amino acids from polypeptides of SEQ ID NO: 1, SEQ ID NO: 2, SEQ ID NO: 3, SEQ ID NO: 4 or SEQ ID NO: 5, where they can be product from full length polypeptide processing. Also, the functional fragment can be produced from expression of truncated nucleic acid sequences SEQ ID NO: 6, SEQ ID NO: 7, SEQ ID NO: 8, SEQ ID NO: 9 and SEQ ID NO: 10.

The polypeptides described herein have the particularity of being composed of high ratios of amino acids that are essential to human and animal nutrition. A food for specified health use (the so-called physiologically functional food) endowed with a function of delivering EAA through addition of EAA giving polypeptides in an organism, can be prepared by using the polypeptide of the invention or the fraction containing the polypeptide as an active component. And the polypeptide of the invention can be used as a food additive of general foods.

The kinds of the above food are not particularly limited. The physiologically functional food may be applicable to any human food, as for example, but not limited to, milk, pudding, curry, stew, meat sauce, ham, cake, chocolate and the like. In particular, milk is preferable since it can facilitate the intake of the polypeptide of the invention which is difficult for infants. Also, the physiologically functional food formed by addition of the polypeptides of the invention may be given to animals under solid or liquid form. Farm animals would particularly benefit of such a food. The amount of the polypeptide of the invention added to the physiologically functional food is appropriately selected depending on the kind of the food, the purpose of addition of the polypeptide of the invention, the effect expected to be produced by the intake of the food, etc.

In one embodiment of the present invention, the polypeptide can be consumed as a nutraceutical compound, i.e. the protein does not require to be consumed as an aliment. The polypeptide can be produced from a cell or an organisms that have dietary properties and further extracted or concentrated to be presented as a pill, a powder, a potion or any other pharmaceutical forms that are commonly no associated to an aliment. The processed EAA-containing polypeptide can therefore be administered to a human or an animal as a food supplement, indistinctively of food consumption.

In one embodiment of the present invention, the animal species may be selected from mammal, birds or fish. In humans, the essential amino acid can be histidine, leucine, threonine, lysine, tryptophan, phenylalanine, valine, methionie or isoleucine, the 9 amino acids that are not synthesized by human cells. However, in humans, as well as in other animal species, methionine, lysine, threonine, leucine, tryptophan, arginine, and analogs or derivatives thereof are the preferred amino acids to be supplied. As a mammal, the human could beneficiate from an amino acid enriched diet in countries where food availability and quality is reduced. As well, in Western countries, excessive intake of fat and sugar is known to cause obesity, hyperlipidemia and the like. The occurrence of different health disorders is linked to feeding with bad food products or by insufficiently equilibrated food. For example, elevations of triglyceride (TG) levels in blood in hyperlipidemia are said to become a cause which brings disorders such as hypertension and arteriosclerosis. Then, a number of attempts to inhibit elevations of TG levels in blood have been made to improve obesity and hyperlipidemia. The issues of being able to keep foods provided to human equilibrated depends most of the time on the bioavailability of the different components thereof. Therefore, food supplementation with high EAA-content proteins would help to keep equilibrated the diet of these populations.

The polypeptides described herein could receive attention for feeding purpose of poultry species as well as other breaded species grown for human consumption purposes. The feed in which the polypeptide of the invention is combined may be either a feed for livestock such as cows, pigs, chickens, etc. or a feed for hatchery fish such as sea breams, young yellowtails, etc.; the kind of the feed is not particularly limited. The amount of the polypeptide of the invention combined in a feed is appropriately selected depending on the kind of the feed, the effect expected to be produced by the intake of the feed, etc. Generally, it is preferable that the polypeptide of the invention be combined in a feed at the ratio of 0.01 to 0.5% by weight.

A feed endowed with a function of delivery of EAA in livestock, etc. can be prepared by combining in a feed the polypeptide of the invention or the fraction containing the polypeptide as an active component.

In the present invention, the limiting amino acids required for milk protein production were particularly targeted, since one application of the invention is food supply for milk cow breeding. Typical dairy cow diets are limiting in the amino acids lysine and methionine. Methionine is important in the synthesis of milk fat, whereas lysine would play an important role in the amino acid metabolism in mammary gland of cow. Previous research showed that milk production is increased by the addition of rumen protected lysine and methionine when feeding the animal with feed proteins. However, adding feed protein also increases the availability of other amino acids and therefore more nitrogen to the animal than needed.

In another embodiment of the present invention, there is provided a new synthetic protein with a biased composition of selected essential amino acids. Synthetic amino acid sequence synthesis allows for a full control of the amino acid composition of the protein and is, thus, an advantage over generating mutations into naturally occurring proteins. To achieve supplementation of food with amino acids, a strategy consisting in the production of high EAA content synthetic polypeptide, such as milk bundle 1 protein (MB-1), has been explored. Characterisation of MB-1 indicated that the design process used results in the stable expression of a new, largely helical protein enriched in the selected essential amino acids (60% in M, T, K and L). After a first round of design, the protein MB-1 was found to have a folded core and low affinity for 8-anilino-1-naphthalenesulfonic acid (ANSA). Its behaviour and expression levels in vivo were found to be far superior to earlier attempts in the area of high essential amino acid polypeptide design. However, investigation of MB-1's properties did also reveal some flaws. The proteins appear to associate into dimers that could dissociate into monomers in the presence of a high salt concentration. As well, its melting temperature was found to be very low (39° C.), and its resistance to proteases at a physiological temperature was also found to be limited, a possible consequence of partial unfolding. As a consequence of these weaknesses, efforts in growing crystals were unsuccessful.

One way for improving the properties of MB-1 is to increase its conformational stability, which often correlates with resistance to proteolytic degradation. To improve MB1's stability, additional design cycle were studied. In the absence of an X-ray-resolved structure, extensive core redesign and elaborated fold-specifying devices had to be ruled out. In order to improve the properties of new proteins or peptides of the present invention, dominant folding principles were identified in selected natural proteins and encoded into the new amino acid sequence. By conferring structure, compactness and stability, a protein normally stable in vivo is designed, regardless of its biased composition. In view of its apparent simplicity, we chose the insertion of disulfide bridges. This strategy was chosen not only for its simplicity but for other reasons as well: (1) an intramolecular disulfide bridge would bring stability to the protein by reducing the entropy of the unfolded state; (2) the disulfide bridge is a well-known stabilizer against proteolysis, probably due to its impact on target access by proteases; (3) its engineering involves only a small modification of the amino acid composition, which is critical in order for MB-1 to remain nutritionally efficient.

According to another embodiment of the present invention, analogs of milk bundle-1 (MB-1) protein have been developed. These analogs were developed to overcome the flaws mentioned above that were observed with MB 1 protein. In a preferred embodiment, five amino acids of the MB-1 protein where replaced by other residues.

The tyrosine at position 62, predicted to be buried in the hydrophobic core, was replaced by a tryptophan. This spectral probe has been useful on two counts: it permitted the confirmation of protein purity using fluorescence, and it has been used as a conformational probe. Replacement of Tyr62 by Trp provides increased hydrophobicity to the protein core, and expand the useful spectroscopic properties of the side chain in position 62. Met10, Leu13, Met87 and Leu91 were also replaced to increase the beneficial properties of MB-1 protein. Cysteins were chosen to replace the amino acids found at the 4 latter positions. Preferentially, mutations at position 13 and 87 are performed within a same mutated MB-1 protein while M10C and L91C are found within the same mutated MB-1 protein, said mutated proteins being trivially named MB-1LH and MB-1RH, respectively. Mutating the amino acids is performed using primer having a nuclei acid sequence comprising SEQ ID NO: 10, SEQ ID NO: 11, SEQ ID NO: 12, SEQ ID NO: 13 SEQ ID NO: 14 for mutation of the amino acids 10, 13, 62, 87 and 91, respectively.

The polypeptides as depicted in SEQ ID NO: 1, SEQ ID NO: 2, SEQ ID NO: 3, SEQ ID NO: 4, and SEQ ID NO: 5 can be separated and purified from a protein occurring in nature. Alternatively, it can be chemically synthesized directly by known methods. It is also possible to prepare the polypeptide of the invention by engineering a gene having a base sequence corresponding to the above polypeptide sequence, inserting the gene into an appropriate expression vector, and expressing the gene in an appropriate host.

In one embodiment of the present invention, there is provided a composition comprising at least one polypeptide having an amino acid sequence selected from the group consisting of SEQ ID NO: 1, SEQ ID NO: 2, SEQ ID NO: 3, SEQ ID NO: 4, and SEQ ID NO: 5 for endowing a human or an animal with at least one essential amino acid by oral administration.

A further embodiment of the present invention is to provide a polypeptide consisting of an amino acid sequence selected from the group consisting of SEQ ID NO: 1, SEQ ID NO: 2, SEQ ID NO: 3, SEQ ID NO: 4 and SEQ ID NO: 5, wherein the latter amino acid sequences are encoded by the nucleotide sequences selected from the group consisting of SEQ ID NO: 6, SEQ ID NO: 7, SEQ ID NO: 8 SEQ ID NO: 9 and SEQ ID NO: 10.

The invention is not limited to these nucleic acid sequences but also extend to all nucleic acid sequences that encodes SEQ ID NO: 1, SEQ ID NO: 2, SEQ ID NO: 3, SEQ ID NO: 4 and SEQ ID NO: 5. These silent variant sequences include those who comprise nucleic acid changes that alter a specific codon, but where the mutant codon encodes the original amino acid. As well, the invention include nucleic acid sequences that encode for the amino acids originally found in SEQ ID NO: 1 to SEQ ID NO: 5, but that are no encoded by codons that are considered as universal. This received particular attention for expression of the polypeptide in organelles such as mitochondria or in other organisms species such as Mycoplasma, Tetrahymena or Euplotes. The invention can be adapted for the particular species in which the polypeptide is expressed. Particularly, the nucleic acid sequences can be adapted to the availability of the different tRNA found among species, to provide the preferred codons for a specific amino acid.

The nucleic acid sequences can be cloned into an expression vector designed for production of a recombinant peptide said recombinant peptide being produced in a bacteria, yeast or an eukaryotic cell. As well, the nucleic acid, or the encoded amino acid sequence, can be found as purified molecules or in a transformed cell. It is therefore an embodiment of the present invention to be provided with a cell transformed with an expression vector or any organism or microorganism having integrated a DNA sequence comprising a nucleic acid sequence selected from the group consisting of SEQ ID NO: 1, SEQ ID NO: 2, SEQ ID NO: 3, SEQ ID NO: 4 and SEQ ID NO: 5.

The present invention will be more readily understood by referring to the following examples which are given to illustrate the invention rather than to limit its scope.

### EXAMPLE I

### Replacement of Tyr-62 by Trp in the Designer Protein Milk Bundle-1 Results in Significant Improvement of Conformational Stability

Materials and Methods

Preparation of MB-1Trp Mutant

Substitution of the Tyr in position 62 by a Trp was performed using the oligo-directed mutagenesis kit <<Altered Sites® II>> (Promega). The mutational oligonucleotide Tyr62-Trp were purified using denaturing polyacrylamide gel electrophoresis (PAGE) and phosphorylated.

(SEQ ID NO:11)

| | |
|---|---|
| MB-1: | 5′-ATG GCC ACT ACG TAC TTC AAA ACG ATG-3′ |

(SEQ ID NO:12)

| | |
|---|---|
| Tyr62Trp: | 5′-ATG GCC ACT ACG TGG TTC AAA ACG ATG-3′ |

The mutation was then confirmed by dideoxynucleotide sequencing using T7 Sequenase™ kit (Amersham Life Science). The mutated MB-1 gene were cloned back in the pCMG20 4-X expression vector and positive clones were checked again by DNA sequencing.

Protein Expression and Purification

Bacteria carrying the mutant vectors were grown at 37° C., 300RPM in 1 L of LB Miller medium (Difco™) to an O.D. of 0.4. Transcription was induced using 1 mM isopropylthio-β-D-galactoside (IPTG) for 3 hours. The cells were then harvested by centrifugation at 3,000×g. The purification procedure was essentially as described bellow. Precipitated cells were resuspended in ice-cold column buffer (10 mM Tris, 200 mM NaCl, 10 mM ethylenediamine-tetraacetate (EDTA), 1 mM sodium azide (NaN₃), pH 7.4). phenylmethylsulfonylfluoride (PMSF), ethylene glycol-bis(β-aminoethylether)-N,N,N′,N′-tetraacetate(EGTA), benzamidine-hydrochloride and benzamide were added to a final concentration of 0.1, 10, 2 and 2 mM, respectively. Cells were then lysed by ten 30-sec sonication pulses using a Branson Sonifier 250 at 60% output control. The sonicated cells were centrifuged at 11,500× g for 30 minutes at 4° C.

The supernatant was then loaded onto a 15 mL amylose column. The maltose binding protein fused with MB-1Trp (MBP-MB-1Trp) was eluted by washing the column with column buffer containing 10 mM maltose (elution buffer). Pooled peak fractions were placed in dialysis tubing (Spectra/Por; MWCO 3,500 Da) with 50 μL factor Xa per 10 mL fusion protein. The bag was placed in 20 mM Tris, 100 mM NaCl, 3 mM CaCl₂ (cleavage buffer) overnight at 4° C. The following morning, the bag was transferred to 10 mM Tris—1 mM EDTA (TE) buffer, pH 8.0. After a 2-hour hour dialysis, the sample was applied to DEAE-Sepharose equilibrated in TE buffer, pH 8.0 (Fast Flow; Pharmacia) and washed with the same buffer. MB-1Trp was collected as the flow-through. The different fractions were analysed for protein content by the bicinchoninic acid (BCA) assay and the positive fractions were pooled and concentrated using BIOMAX-5K concentrators (Millipore™). Protein samples were prepared in a borate-phosphate buffer (55 mM NaH₂PO₄, 35 mM Na₂B₄O₇, pH 6.8) and dyalised overnight against this buffer prior to measurements. Protein concentration was adjusted to 0.4 mg/mL unless specified otherwise.

Protein Quantification and Electrophoresis

Protein concentration was determined by the BCA assay (Sigma), using bovine serum albumin as the standard. The protein was visualised by sodium dodecyl sulfate polyacrylamide gel electrophoresis (SDS-PAGE) using 12% polyacrylamide-tricine gels, followed by silver nitrate staining. SDS-PAGE experiments were conducted prior to measurements to confirm protein purity.

Conformational Investigation by Circular Dicroism (CD)

Protein samples were degassed for 20 minutes at 20° C. before measurements. Spectra were measured with a Jasco™ J-720 spectropolarimeter, which was routinely calibrated with a 0.06% (W/V) ammonium (+)-10-camphorsulfonate solution. For measurements in the far-UV region a quartz cell with a path length of 0.01 cm was used. Ten scans were accumulated at a scan speed of 20 nm per minute, with data being collected at every nm from 180 to 260 nm. Sample temperature was maintained at 20° C. using a Neslab™ RTE-111 circulating water bath connected to the water-jacketed quartz cuvettes. Spectra were corrected for buffer signal and conversion to Δε (on the basis of amide bond concentration) was performed with the Jasco™ Standard Analysis software. Secondary structure calculations were performed using the CDsstr program using default settings.

Thermal Denaturation

Samples were prepared as described in the preceeding section. In order to measure thermostability, temperature was increased from 15 to 85° C. at a rate of 30° C. per hour using a Neslab™ RTE-11 controlled by the Jasco™ spectropolarimeter software. CD spectra were collected at every 5° C., from 200 to 260 nm, at a scan speed of 20 nm/min. In order to assess reversibility of thermal denaturation, the protein solutions were cooled down at a rate of 30° C. per hour, and spectra were measured at 70, 50 and 20 ° C.

Thermal stability was calculated assuming a unimolecular, two-state process as described bellow. The Δε_{MRW} at 222 nm measured at various temperature was used as the property (y) indicative of the extent of unfolding. In the folded state, the parameter y=y_{f} and the fraction of folded protein f_{f} is equal to 1. When the protein is unfolded, the parameter y=Yᵤ, and the fraction of unfolded protein fᵤ is equal to 1. For intermediate states, y is given by y_{f}f_{f}+yᵤfᵤ. Thus, by measuring y, we can calculate the fraction of protein unfolded: fᵤ=(y_{f}−y)/(y_{f}−yᵤ). The equilibrium constant for the unfolding process is Kᵤ=fᵤ/(1−fᵤ) and melting temperatures (Tₘ) are obtained at Kᵤ=1.

ANSAF Fluorescence Enhancement

Protein concentration was adjusted to 0.1 mg/mL in B/P pH 6.8 and equilibrated at room temperature (RT) for 1 hour. Then ANSA was added to a final concentration of 10 μM and equilibrated 5 minutes prior to measurements. Spectra were recorded using an LS50-B Perkin-Elmer™ fluorometer with an excitation wavelength of 380 nm. Spectra were collected from 410 to 550 nm. Correction for buffer signal on ANSA was keyed in when applicable.

Intrinsic Fluorescence Measurements

Protein concentration of samples was adjusted to 0.1 mg/mL and equilibrated at room temperature (RT) for 1 hour. For chemical denaturation, Urea (Sigma U-5378) was added to a final concentration of 8M and equilibrated at RT 5 minutes before measurement. For thermal denaturation, temperature was increased to 85° C. at a rate of 15° C. per hour using Neslab™ RTE-11. Spectra were recorded using an L550-B Perkin-Elmer™ fluorometer with an excitation wavelength of 280 nm. Spectra were then corrected for buffer signal before analysis.

Size-Exclusion Chromatography/Estimation of Molecular Size

Protein samples were adjusted to a concentration of 0.6 mg/mL and loaded onto a Beckmann Ultraspherogel SEC-2000™ column hooked to a Waters 510 HPLC. The column was standardized with the Pharmacia Low Molecular Weight gel filtration calibration standards prepared in the same buffer.

Proteolytic Degradation Measurements

Experiments were carried out as described bellow. Reactions were stopped by the addition of 2% SDS buffer followed by heating for 3 minutes at 100° C. Samples were then analysed on SDS-PAGE followed by silver nitrate staining. The amount of protein remaining after incubation with proteases was determined by measuring the optical density of each band using the image analysis system Imaging Research MCID. Cytochrome c from horse heart (Sigma C-7752) was used as a reference protein for all degradation runs in order to correct for possible variations in crude proteolytic activity.

Results

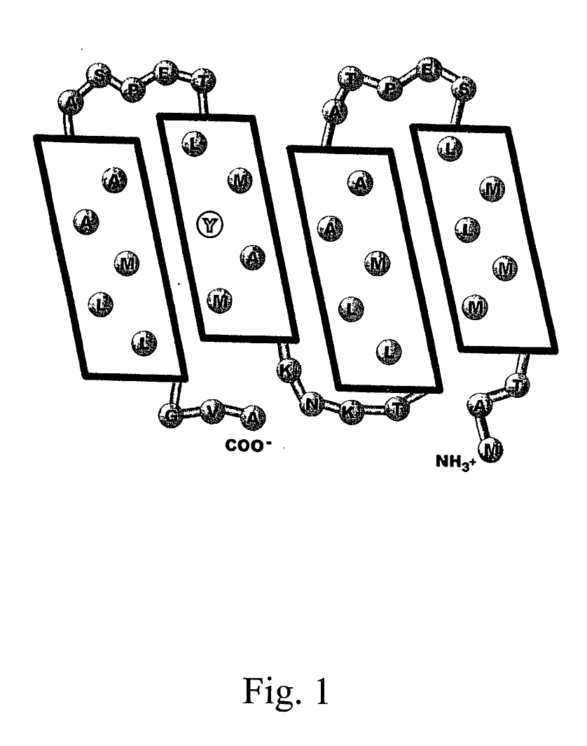

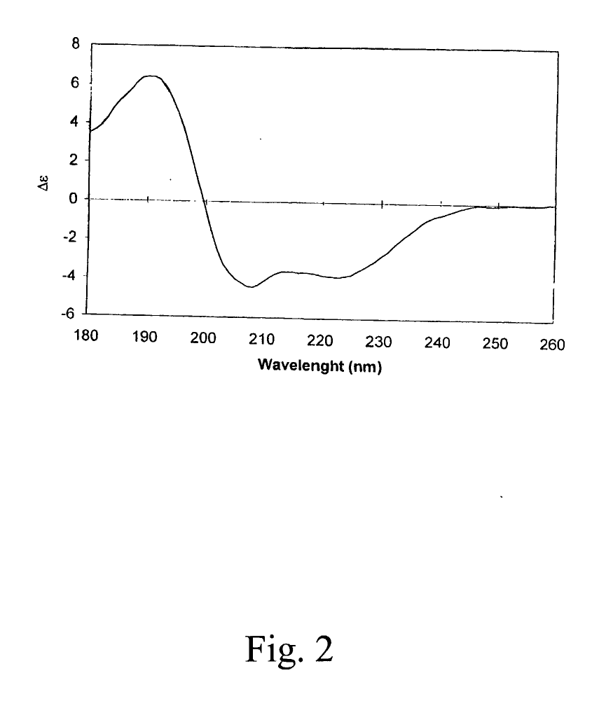

Position 62 in MB-1 was chosen for the emplacement of a spectroscopic probe at the moment of initial design. As shown on the model in FIG. 1, this position is part of the hydrophobic core, and a niche made of 5 Ala was built around it in order to accommodate a larger side chain in this region of the core. Substitution of Tyr62 for Trp was performed on MB-1 by site-directed mutagenesis and confirmed by DNA sequencing and fluorospectroscopy; then the mutant, named MB-1Trp, was characterized. First, circular dichroism (CD) measurements were performed and spectra typical of helical proteins were obtained (FIG. 2). Using CDsstr algorithm, 50% of amide groups were predicted to be in a helical environment. Thus, the secondary structures of the mutant MB-1Trp were found to be similar to the parent molecule MB-1.

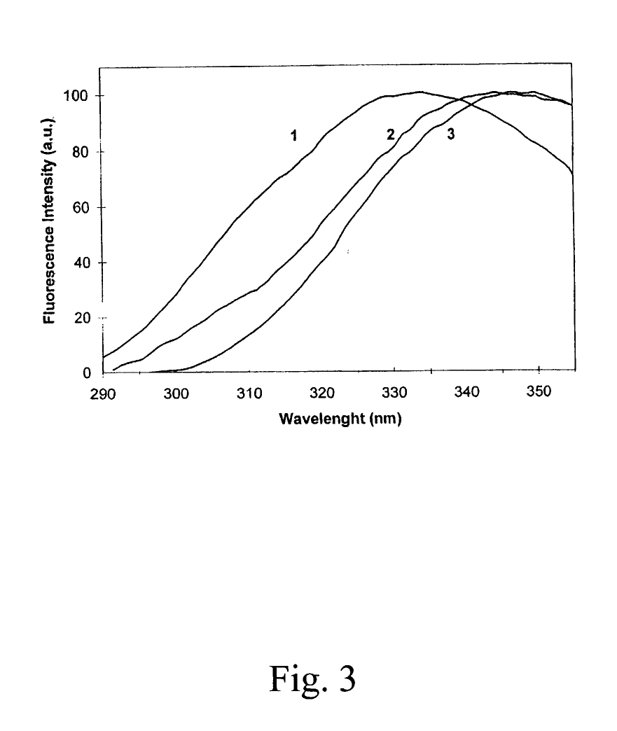

The formation of a tertiary structure in the mutant was confirmed by fluorescence measurements: the folded protein fluorescence peaked at 333 nm, and its emission shifted to approximately 345 nm when unfolded using heat or urea (FIG. 3). The Trp side chain appears to be protected in the folded protein, and exposed to solvent upon unfolding, as predicted by design. Similar shifts in fluorescence λₘₐₓ were observed for another designer protein (α4) after insertion of a Trp side chain.

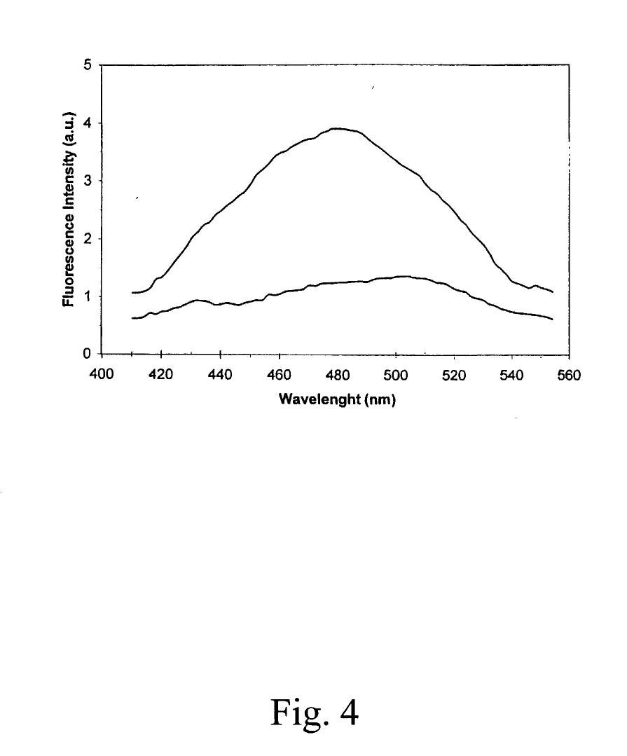

Results from fluorescence measurements using the probe ANSA are shown in FIG. 4. The enhancement of ANSA emission by the proteins was limited to a factor of 3, which is comparable to the parent molecule, and lower than the values expected for poorly folded proteins. These results suggest that MB-1Trp is rather well folded, and not in a molten globule state. Size-exclusion chromatography analysis revealed that MB-1Trp migrated as a 12 kDa protein, closed to the expected size of an MB-1Trp monomer. Substitution of Trp in position 62 appears to correct one weakness of the initial design of MB-1, which had a size approaching that of a dimer.

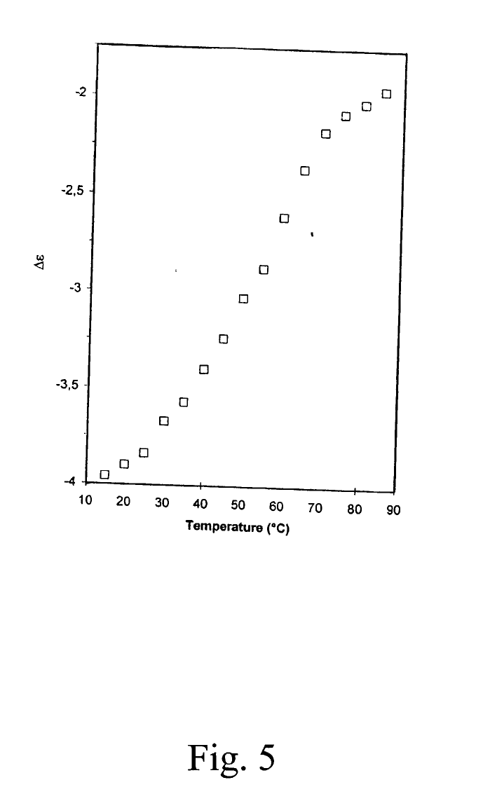

The impact of the substitution in position 62 on conformational stability was verified using two different approaches. First, the CD signal at 222 nm was recorded at various temperatures in order to monitor unfolding of MB-1Trp helices. The denaturation of MB-1Trp (FIG. 5) indicated a melting temperature of 55° C., a significant improvement over MB-1 (melting temperature of 39° C.) and other mutants characterized so far. The thermal denaturation was found to be fully reversible, another improvement over MB-1, but the transition was spread over a wide temperature range (30° C. to 65° C.). However, this apparent lack of cooperation in MB-1Trp thermal unfolding also characterized MB-1 and other small designer proteins.

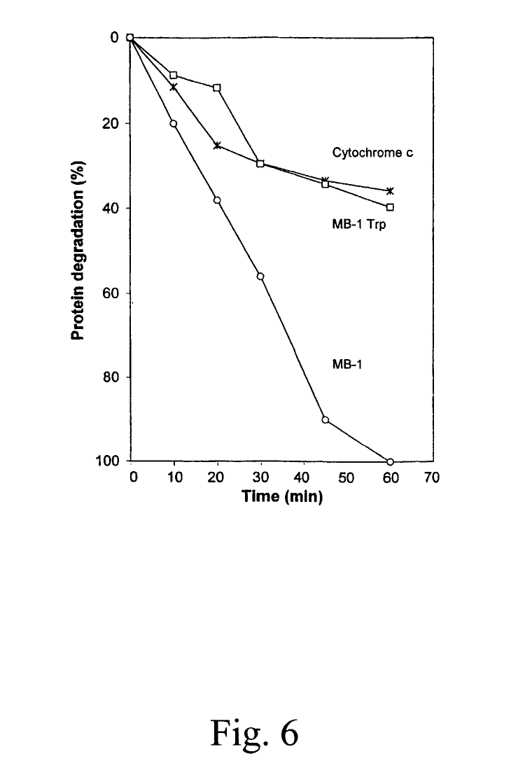

Stabilization of MB-1 fold by the mutation was also confirmed by proteolytic degradation experiments. Degradation curves shown in FIG. 6 clearly demonstrate a gain in resistance to proteolytic attack. Under standard conditions, MB-1Trp was twice as resistant as MB-1, and behaved like a natural protein of similar size (cytochrome C).

Discussion

Trp and Tyr residues are comparable on various counts: they have large aromatic side chains, they undergo a limited but similar loss of conformational entropy upon protein folding; and they have similar secondary structure propensities. They differ in their hydrophobicities however, due to the presence of a hydroxyl group in p position on the phenyl moiety. When such a polar group is involved in a hydrogen bond, the difference between Trp and Tyr hydrophobicity decreases, but in MB-1, no such H-bonding partner was properly positioned while designing the protein. Most results shown here indicate that the increase in hydrophobicity in position 62, due to the removal of tyrosine's hydroxyl group from the core, led to an important improvement of MB-1 fold stability. This may be explained by tryptophan's ability to make extensive van der Waals contacts with neighboring residues due to its large size, in addition to its contribution to the hydrophobic effect. The improvement of MB-1 properties also included the specification of a monomeric organization.

Trp and Tyr both belong to the class of large side chains, but Trp is significantly larger than Tyr, with an additional 34 cubic Angstroms. The original design strategy allowed for a Tyr in position 62, but not for Trp, which would result in a layer volume that would be above average for natural bundles. The results shown here all indicate that MB-1 secondary and tertiary structures were not disturbed by the substitution Tyr62-Trp. The protein is helical and well folded and it appears that the niche around position 62 readily accommodates the larger side chain of Trp. The mutation resulted in an important improvement in the protein stability

The gain in conformational stability afforded by the Tyr62-Trp mutation is of paramount importance for future advances in the development of MB-1 family of proteins. Its high resistance to proteases permit the production of this high quality protein by transgenic crops to be used in animal production. A comparison of previously reported analyses of plant protein degradability indicates that MB-1Trp compares to sunflower 2S seed albumin 8 protein, a protein with a high methionine content intended for production of transgenic crops with enhanced nutritional quality. Note that the EAA profile of SFA8 is not optimized for lactating cows needs, while MB-1Trp is, due to its balanced content of methionine, lysine and threonine. This comparison indicates that although few cycles of design may be required, our design approach produced a high quality protein that competes with natural proteins at the level of stability.

The mutant presented here clearly outperforms the parent molecule on several counts, including the ability to stay monomeric under conditions used here, and to resist degradation in solution.

### EXAMPLE II

### Engineering Nutritious Proteins: Improvement of Stability in the Designer Protein MB-1 Via Introduction of Disulfide Bridges

Materials and Methods

Preparation of the New Mutants

Substitution of Cys in position 13 and 87 of MB-1 (MB-1LH) and positions 10 and 91 of MB-1 (MB-1RH) was performed using the oligo-directed mutagenesis kit << Altered Sites® II>> (Promega). The mutational oligonucleotides (1 for position 13; 2 for position 87, 3 for position 10; 4 for position 91 shown below with the corresponding MB-1 sequences) were purchased from GibcoBRL/Life Technologies™, purified using denaturing polyacrylamide gel electrophoresis (PAGE) and phosphorylated.

| | | | |
|---|---|---|---|
| | MB-1: | 5′-ATG ATG ACC ACC CTG TTT AAA ACT ATG-3′ | (SEQ ID NO:13) |
| Oligo 1: | L13C: | 5′-ATG ATG ACC ACC TGC TTT AAA ACT ATG-3′ | (SEQ ID NO:14) |
| | MB-1: | 5′-ACG GCT ACA ACC ATG AAA AAT CAT CTG-3′ | (SEQ ID NO:15) |
| Oligo 2: | M87C: | 5′-ACG GCT ACA ACC TGC AAA AAT CAT CTG-3′ | (SEQ ID NO:16) |
| | MB-1: | 5′-ATG ACC GAC ATG ATG ACC ACC CTG TTT-3′ | (SEQ ID NO:17) |
| Oligo 3: | M10C: | 5′-ATG ACC GAC ATG TGT ACC ACC CTG TTT-3′ | (SEQ ID NO:18) |
| | MB-1: | 5′-ATG AAA AAT CAT CTG CAG AAC TTG ATG-3′ | (SEQ ID NO:19) |
| Oligo 4: | L91C: | 5′-ATG AAA AAT CAT TGC CAG AAC TTG ATG-3′ | (SEQ ID NO:20) |
| | MG-1: | 5′-ATG GCC ACT ACG TAC TTC AAA ACG-3′ | (SEQ ID NO:21) |
| Oligo 5: | Y62W: | 5′-ATG GCC ACT ACG TGG TTC AAA ACG-3′ | (SEQ ID NO:22) |

The Tyr62-Trp mutation lead to MB-1Trp and was achieved directly in the expression vector pMal-c2. The mutational oligonucleotide 5 was used and treated as described above. The mutations were then confirmed by dideoxynucleotide sequencing using T7 Sequenase kit (Amersham Life Science). The mutated MB-1 genes were cloned back in pCMG20 4-X, the expression vector. All positive clones were checked again by DNA sequencing after cloning.

Protein Expression and Purification

All mutated proteins were prepared as described in Example I. After purification, all proteins were checked for purity by sodium dodecyl sulfate polyacrylamide gel electrophoresis (SDS-PAGE) (protein purity>95%).

Protein Quantification and Electrophoresis

Protein concentration was determined by the bicinchoninic acid (BCA) assay (Sigma), using bovine serum albumin as standard. The protein was visualised by sodium dodecyl sulfate polyacrylamide gel electrophoresis (SDS-PAGE) using 12% polyacrylamide-tricine gels, followed by silver nitrate staining. SDS-PAGE experiments were conducted prior to measurements to confirm protein purity.

Quantification of Cys Residues and Assessment of Bridge Formation

Chemical modification of the Cys residues by iodoacetamide and iodoacetate was performed as described bellow. Comparison of the electrophoretic mobility of the reduced and non-reduced form of the MB-1 mutant was done by SDS-PAGE. The Cys were also quantitated by reacting with p-hydroxymercurybenzoate (p-HMB) and measuring absorbance of p-HMB at 260 nm with a UV/vis spectrophtometer (Novaspec™, Pharmacia).

Conformational Investigation by Circular Dichroism (CD)

Unless specified otherwise, protein samples were prepared at a concentration of 0.6 mg/mL with a phosphate buffer (128 mM NaH₂PO₄, pH 6.8). The reductive agent dithiothreitol (DTT) was added to a final concentration of 10 mM where specified. The samples were then degassed and equilibrated 20 minutes at 20° C. before measurements. Spectra were measured with a Jasco™ J-720 spectropolarimeter, which was routinely calibrated with a 0.06% (w/v) ammonium (+)-10-camphorsulfonate solution. For measurements in the far-UV region, a quartz cell with a path length of 0.01 cm was used. Ten scans were accumulated at a scan speed of 20 nm per minutes, with data being collected at every nm from 180 to 260 nm. Sample temperature was maintained at 20° C. using a Neslab™ RTE-111 circulating water bath connected to the water-jacketed quartz cuvette. Spectra were corrected for buffer signal and conversion to Δε_{MRW} (mean residue weight) was performed with the Jasco Standard Analysis software. Secondary structure calculations were performed using the CDsstr program using default settings.

Thermal Denaturation

Samples were prepared as described in the preceding section. In order to measure thermostability, temperature was increased from 10 to 95° C. at a rate of 30° C. per hour using a Neslab™ RTE-11 controlled by the Jasco spectropolarimeter software. CD spectra were collected at every 5° C., from 200 to 260 nm, at a scan speed of 20 nm/min and CD signal at 222 nm were collected at 1° C. interval. In order to assess reversibility of thermal denaturation, the protein solutions were cooled down at a rate of 30° C. per hour, and spectra were measured at 70, 50 and 20° C.

Thermal stability was calculated assuming a unimolecular, two-state process as previously described. The CD signal at 222 nm at various temperature was used as the property (y) indicative of the extent of unfolding. In the folded state, the parameter y=y_{f} and the fraction of folded protein f_{f} is equal to 1. When the protein is unfolded, the parameter y=Yᵤ, and the fraction of unfolded protein fᵤ is equal to 1. For intermediate states, y is given by y_{f}f_{f}+Yᵤfᵤ. Thus, by measuring y, we can calculate the fraction of protein unfolded: fᵤ=(y_{f}−y)/(y_{f}−yᵤ). The equilibrium constant for the unfolding process is Kᵤ=fᵤ/(1−fᵤ) and melting temperatures (T_{M}) are obtained at Kᵤ=1.

8-Anilinonaphthalenesulfonic Acid (ANSA) Fluorescence Enhancement

Protein concentration was adjusted to 0.2 mg/mL and equilibrated at room temperature (RT) for 1 hour. Then ANSA was added to a final concentration of 10 μM and equilibrated 5 minutes prior to measurements. Spectra were recorded using an LS50-B Perkin-Elmer™ fluorometer with an excitation wavelength of 380 nm. Spectra were collected from 410 to 550 nm. Correction for buffer signal and for the effect of DTT on ANSA was keyed in when applicable.

Fluorescence Quenching Measurements

Protein concentration of samples was adjusted to 0.2 mg/mL and equilibrated at RT for 1 hour. Where specified DTT was added to a final concentration of 10 mM and equilibrated at RT 15 minutes before adding the quencher citrate (final concentration 0.25 M). Control samples were exposed to 0.75 M NaCl in order to keep the same ionic strength as in the samples exposed to 0.25 M citrate. Mutants containing Tyr were excited at 280 nm and fluorescence spectra were recorded from 290 to 330 nm. For the Trp mutants, fluorescence was collected from 310-400 nm. Quenching of the Trp mutants was performed with 0.3M potassium iodate, and NaCl was used in controls. Spectra were then corrected for buffer signal before analysis. The effect of DTT on fluorescence was corrected when needed.

Proteolytic Degradation Measurements

Proteins were dialysed against 2000 volumes of borate-phosphate buffer (pH 6.8) at 4° C. overnight. Experiments were then carried out as described bellow. Reactions were stopped by the addition of 2% SDS buffer followed by heating for 3 minutes at 100° C. Samples were then analysed on SDS-PAGE followed by silver nitrate staining. The amount of protein remaining after incubation with proteases was determined by measuring the optical density of each band using the image analysis system Imaging Research MCID. Cytochrome C (horse heart, Sigma) was used as a reference protein for all degradation runs in order to correct for possible variations in crude proteolytic activity.

Size Exclusion Chromatography (SEC)

Proteins were applied to a Beckman Ultraspherogel SEC-200 column at a concentration of 0.6 mg/mL. A constant flow of 0.8 mL/min was maintained using a Waters HPLC pump. Pharmacia Low Molecular Weight gel filtration calibration standards were used for column calibration.

Results

Design Strategy

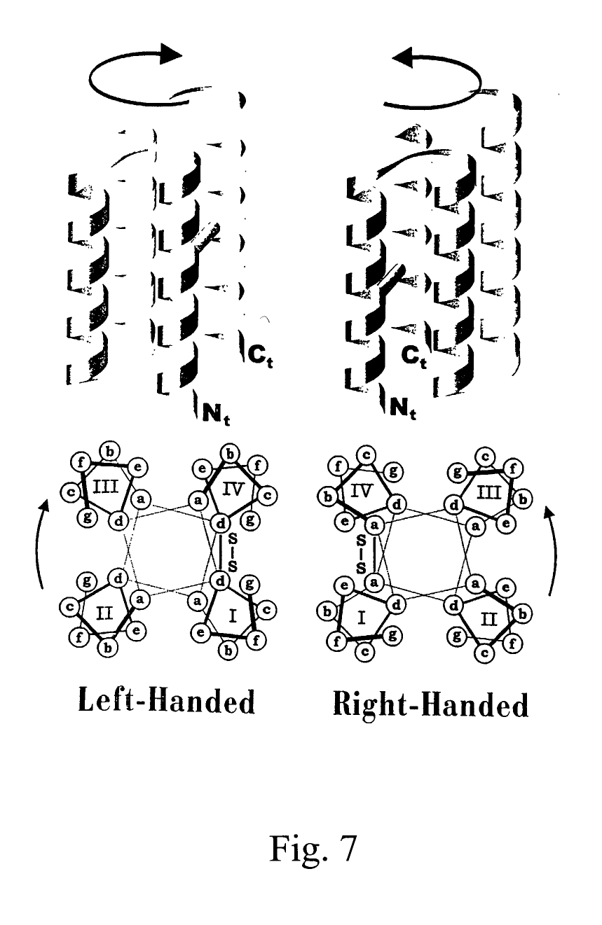

The putative modifications to MB-1 structure are illustrated in FIG. 7. The design strategy used here focused on two aspects: 1—the restrictive effect of a covalent bond between remote residues on the protein as a whole; and 2—the precise location of Cys which permits disulfide bridge formation. By choosing positions as far apart as possible, one can reduce the entropy gain upon unfolding for most of the protein. Thus, insertion of a bridge between helices I and IV would enclose a larger part of the polypolypeptide than a bridge involving other helices. Another consideration for using helix I is that this MB-1 segment of sequence is sensitive to proteolytic degradation. The restriction of helix I by Cys insertion could help prevent such a phenomenon.

The position of Cys in helices I and IV must allow sulfhydryl groups to be properly aligned in order to minimise strain induced by bridge formation. On the basis of geometric models built for similar proteins, it appeared that position “d” of the heptad pattern used for MB-1 design would offer the best geometry for bridge formation. Therefore, L13 and M87 residues were selected for mutation to Cys. FIG. 7 depicts the expected location of the bridge in the mutant (hereafter referred to as MB-1LH, assuming it folds as per design). Note that for proper alignment of position “d” in helices I and IV, a left-hand connectivity of the helices had to be assumed (i.e. the bundles are positioned such that when helix I is at the fore front, with its N-terminus pointing down, then helix II is placed to the left of helix I). A second scenario was considered, in which a right-hand connectivity could be specified. Examination of the second model in FIG. 7 suggests mutations at positions “a” in helices I and IV, since positions “d” would be too far apart. By choosing M10 and L91 residues for mutation to Cys, we attempted to generate a mutant (named MB-1RH) that would resemble MB-1LH as much as possible, except for reversing its connectivity.

Disulfide bridges was also inserted into MB-1Trp. This protein is a derivative of MB-1 where Tyr62 was replaced by Trp. Position 62 in MB-1 was chosen for the emplacement of a spectroscopic probe at the moment of initial design. As shown on the model in FIG. 1, position 62 is part of the hydrophobic core, and a niche made of 5 Ala was built around it in order to accommodate a larger side chain in this region of the core. The replacement of Tyr by Trp was thought to improve on stability, and indeed, characterisation of MB-1Trp confirmed the strategy. MB-1Trp has a melting temperature of 55° C. and is more resistant to protease action than MB-1. Here we are going to use MB-1Trp because of the increase in bulk offered by Trp in the core, in a way to compensate for the loss of volume consequent to the mutations used for bridge insertion.

Initial Characterisation of Mutant Structure

The presence of two Cys in all mutants was confirmed by Cys derivatization with mixtures of iodoacetamide and iodoacetate, and by reacting Cys with p-HMB, in agreement with the DNA sequences of the expression vectors. Formation of the bridges was confirmed by a comparison of the protein migration after and before treatment with DTT, a reductive agent. Reduction of the protein changed its migration speed, and the impact of reduction appeared to modify mobility of all proteins treated. Replacement of Tyr by Trp was confirmed by spectrofluorometry.

Secondary Structures Analysis

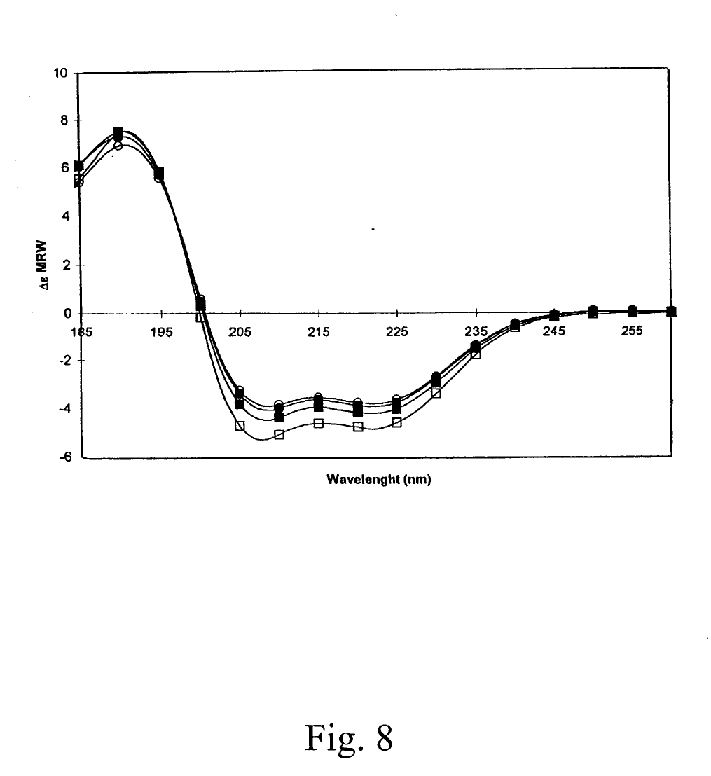

Secondary structure analysis of the mutant was performed using circular dichroism (FIG. 8, Symbols are: open circles for MB-1LH; open squares for MB-1RH; closed circles for MB-1TrpLH; and closed squares for MB-1TrpRh). The spectra measured for all mutants with their bridge closed (no DTT) were typical of helical proteins, and calculations of helical amide contents were in the range expected for these proteins (Table 1). MB-1LH, MB-1RH and MB-1TrpLH appeared to have similar secondary structures as their parent molecules MB-1 and MB-1Trp (which contain approximately 50% helical amides. The protein MB-1 appeared to be tolerant to bridge insertion, regardless of the connectivity imposed by this bridge (either handedness).

Inserting the bridges leading to a right-hand connectivity into MB-1Trp led to a different scenario: the helical content of MB-1TrpRH differed significantly in secondary structure contents, with only 42% helical content (Table 1). The right-hand connectivity in MB-1TrpRH imposes a different conformation, with less helical content.

TABLE 1
Comparison of physical properties measured for the four mutants

| | α- | Monomer | Monomer | | |
|---|---|---|---|---|---|
| | helix^{a} | population | population SDS- | | Protein |
| | +/−3 | SEC^{b} | PAGE^{c} | Tm^{d} | undegraded^{e} |
| Mutant | % | +/−5% | +/−5% | +/−1° C. | +/−8% |
| RH | 50 | 100 | 100 | 50 | 35 |
| | (23)^{f} | | | | |
| LH | 48 | 100 | 100 | 48 | 43 |
| | (18) | | | | |
| MB- | 42 | 90 | 95 | 49 | 40 |
| 1TrpRH | (25) | | | | |
| MB- | 55 | 100 | 100 | 42 | 17 |
| 1TrpLH | (34) | | | | |

^{a}Helix percentage calculated with CD spectra.
^{b}Oligomerization population estimated from exclusion chromatography.
^{c}Oligomerization population estimated from SDS-PAGE gel analysis
^{d}Melting temperature derived from thermal denaturation.
^{e}Percentage of protein undegraded after a 60 minutes of incubation with pronase E.
^{f}Numbers in parenthesis are for proteins with opened disulphide bridge (treated with DTT).

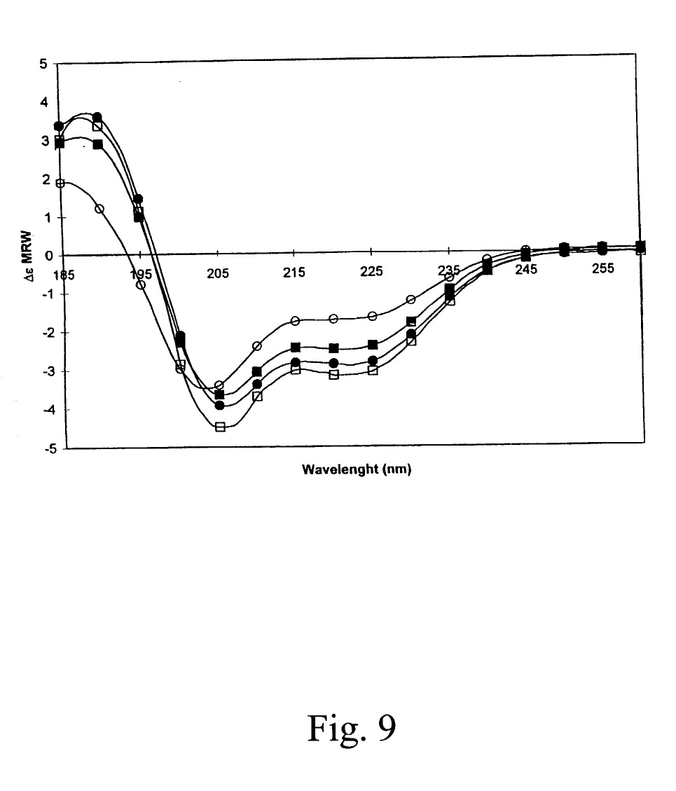

The mutants were then analysed in the presence of DTT (FIG. 9, Symbols are: open circles for MB-1LH; open squares for MB-1RH; closed circles for MB-1TrpLH; and closed squares for MB-1TrpRh), in order to separately assess the effect of the mutations (insertion of Cys) from the impact of bridge formation. All mutants helical contents dropped when exposed to DTT as indicated by a loss of signal intensity at 190 and 225 nm, with MB-1LH being nearly completely unfolded (Table 1). This suggests that in the absence of the bridge, mutations used here are destabilising. Among the four mutants presented here, MB-1LH had the largest drop in helical content after bridge opening (down to 18%, see Table 1). This means that the mutations M13-Cys and L87-Cys promote unfolding of the protein, and that formation of the bridge compensates for this deleterious effect.

When the same treatment was performed on MB-1TrpLH, the loss of helical content was less severe (down to 34%) (Table 1). The fact that MB-1TrpLH stay somewhat better folded (34%) than MB-1LH in the presence of DTT may suggest that Trp compensate for the loss of bulk consequent to Cys insertion in the core with a left-hand connectivity. Partial unfolding after DTT treatment was confirmed by fluorescence quenching and ANSA binding for all mutants.

Reversibility of DTT-induced unfolding was verified by CD measurements. After DTT removal, CD spectra were similar to the native spectra, suggesting that all four proteins refolded after bridge repair (FIG. 8).

Quaternary Structure

The ability to control protein association and aggregation has been (and still is) an important tumble stone in de novo protein design. The protein association was monitored for the mutants under two sets of conditions. First, quaternary organisation was monitored by SEC under benign buffer conditions in a way to observe the impact of both covalent and non-covalent bonds. Then, the proteins were denatured and migrated on an SDS-PAGE gel in order to detect any inter-molecular covalent bond. Results in Table 1 indicate that all mutants, except MB-1TrpRH, were monomeric, regardless of the treatments used here. This confirms that intra-molecular bridges are formed, and suggest that the three proteins fold as planned. At variance, MB-1Trp RH was found to contain 5-10% dimer that resisted SDS treatment, which indicate that these dimers are formed via an inter-molecular bridges. Other oligomerization states have been observed for this protein under specific experimental conditions. An increase in surface hydrophobicity was detected using ANSA binding measurements (ratio I₄₈₀/I₅₁₀ for MB-1TrpRH=12, compared to a ratio of 7-8 for the other three mutants), which indicate that MB-1TrpRH core is more fluid, allowing for inter-molecular bridges.

Effect of the Mutations on Conformational Stability

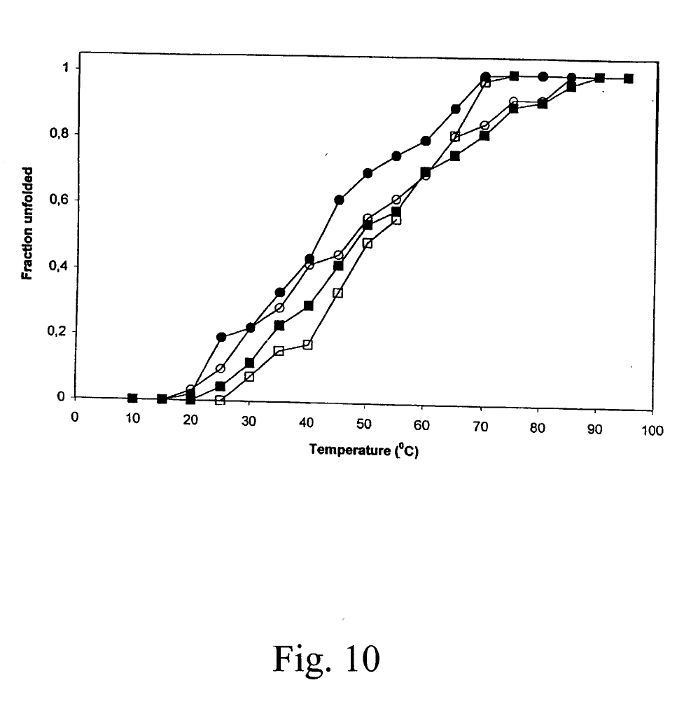

Conformational stability of both mutants was measured using CD as described in Materials and Methods. FIG. 10 shows the four denaturation curves, while calculated Tm values are listed in Table 1. Both MB-1 mutants clearly outperform MB-1 (Tm=39° C. (24)), by approximately 10° C. in thermostability. Symbols are: open circles for MB-1LH; open squares for MB-1RH; closed circles for MB-1TrpLH; and closed squares for MB-1TrpRh. Approximately 100% of the helical content was recovered after denaturing the protein by cooling to room temperature as ed earlier, whereas the MB-1 parent molecule could not be refolded under similar experimental conditions.

Insertion of bridges into MB-1Trp resulted in a completely different condition, both mutants being less stable than their ancestor molecule MB-1Trp (Tm=55° C.), with MB-1TrpLH being the least stable (TM=42° C.). The mutants seemed less stable than the pair MB-1LH and RH.

Effect of the Mutations on Proteolytic Degradation

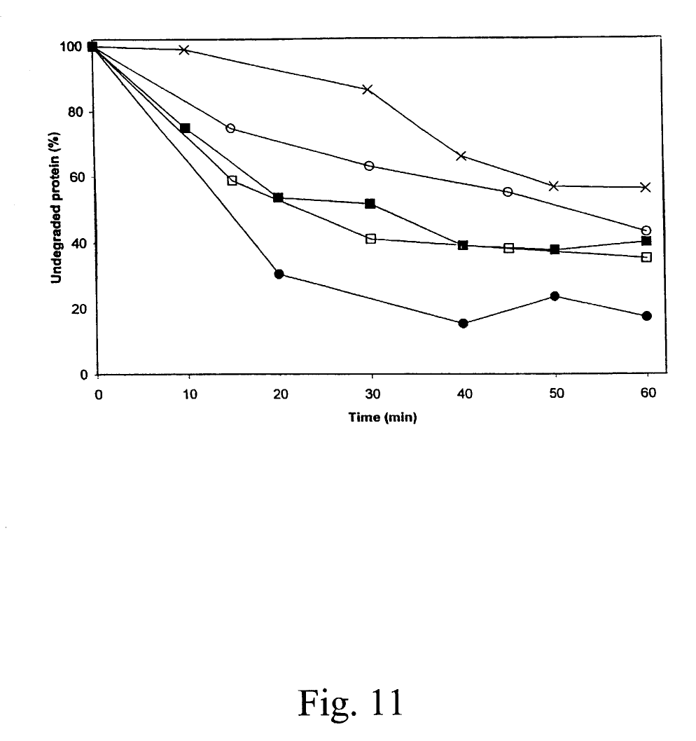

Proteolytic degradation experiments were carried out as ed in FIG. 11. Symbols are: open circles for MB-1LH; open squares for MB-1RH; closed circles for MB-1TrpLH; and closed squares for MB-1TrpRh. Both mutants derived from MB-1 outperformed MB-1, with about 40% intact protein left after a 1 hour treatment with proteases (MB-1 cannot be detected after same treatment). MB-1LH is more resistant than MB-1RH first 45 min, but the difference decreases after 60 min. The mutants derived from MB-1Trp were less stable than their parent molecule (60% intact MB-1Trp left under same conditions). MB-1TrpRH behaved like MB-1RH and the other MB-1 mutants, while MB-1TrpLH has the lowest resistance, with 17% proteins left after treatment (Table 1). Once again, the insertion of Trp near the bridge did not lead to any improvement on stability. Degradations were performed in the presence of DTT and higher degradation rates were measured. However, difficulties were encountered due to the inhibitory effect of DTT on the proteolytic activity and the need to prevent re-oxidation of the disulfide bridge, thus strongly limiting the reliability of these data.

Discussion

The insertion of disulfide bridges in MB-1 was found to have an important impact on protein behaviour. Both mutant proteins were more resistant and stable than MB-1. Their unfolding (be it after bridge reduction or due to high temperature) was found to be reversible, and their apparent size in solution indicates they are monomeric. Both strategies led to serious behaviour improvement when compared to the MB-1 parent molecule, in accordance to the impact of disulphides on protein resistance. This phenomenon cannot be ascribed to Cys mutations per se (which are destabilising), but is a consequence of specifying connectivity and limiting protein skeleton freedom through disulfide bond formation.

Predicting (and understanding) the impact of Cys mutations separately from the effect of bridge formation have been difficult in the past. As a result, engineering disulfides in natural proteins have sometimes led to loss of stability. Separately monitoring the effect of a mutation from that of bridge formation was performed. Results obtained in the presence of DTT indicate that Cys mutations are destabilizing for both proteins. A negative impact of Cys insertion in heptad positions “a” and “d” may be observed. Cys is a poor helix former, it is smaller than Met or Leu, and its polarity is higher than that of Met or Leu.

Closing the bridge led to an increase in stability, which suggests that possible strain induced by cross-linking helix I and IV may be less important. Thus, the geometry and the position of the Cys residues appear to be adequate for bridge formation as expected per design.

In MB-1Trp, the core Tyr62 has been replaced by Trp, leading to an increase in hydrophobicity and bulk in the core. Thus, we attempted to compensate the impact of the Cys mutations by using MB-1Trp for target. The disulfide bridged constructions harbouring Trp 62 analysed here were not better behaved than the ones based on MB-1. MB-1TrpRH appeared to form concatemers linked by inter-molecular bridges, indicating a high conformational flexibility. MB-1 TrpLH had the lowest resistance to proteases, suggesting some undetected flaw in folding. Therefore, the insertion of Trp in position 62 of bridged MB-1 protein is not appropriate, and one might infer that this position is too close to the bridges, promoting steric hindrance.

The mutations used here allowed to increase MB-1 stability and resistance to proteases, which improves MB-1 analogs or mutants efficiency as a food additive.

### EXAMPLE III

### Controlled Polymerization of the Synthetic Polypeptide MB-1TrpRH

Formation of inter-molecular disulfide bridges between protein monomers may allow to expand the range of applications for such a protein. Polymerization contributes to protein stability. The proximity of proteins attached into a polymer excludes large proteases and allow for protection of otherwise exposed targets. Polymerisation would thus improve on production yield in a given in vivo environment (bacteria or plants) and improve on the performances of the polypeptide when used as a feed additive in polygastric animals such as cows. Polymerising proteins may also be of interest in molecular biology diagnostics. For example, SDS-PAGE protein standards are often made of a mixture of different proteins, with different staining properties.

As a results, stain density may vary greatly from one protein to the next. Further, most protein standard kits include proteins with size that may not cover the range of sizes to be measured in a regular manner. A solution to these limitations is a ladder made of similar proteins, separated by a fixed increment when going from a band to the other. Such a ladder is actually available on the market, and is made of proteins obtained from genetic constructions where an increasing number of coding regions are fused (BioRad™, Precision Protein Standard) in one gene. In this kit, proteins of different sizes are produced by different transformed microbes producing monomer, dimers, trimers, and higher oligomers of the same subunit (monomer), extracted and mixed in proper amounts in order to serve as molecular weight markers. Since one transformed organism encodes only one association state (i.e. one clone produces dimers, the other one expresses trimers, etc.), a number of organisms have to be maintained, grown, harvested in order to produce protein of different sizes. Here we report a protein ladder produced by one transformed organism, which may simplify the preparation of protein molecular weight markers.

Materials and Methods

The protein used as the monomer (or repeating unit) is a mutant of the designer protein MB-1 where the mutations Met10-Cys, Leu91-Cys and Tyr62-Trp were performed by oligonucleotide site-directed mutagenesis. The production by Escherichia coli and purification protocol is as reported in Morrison et al. 2000, except for few modifications. A salt gradient was used (0 to 50 mM NaCl) in order to elute the proteins from a DEAE Sepharose column. Fraction containing the mutant MB-1TrpRH were collected and concentrated using Millipore concentrators. The proteins were then incubated in 10 mM DTT for 8 h at 4° C. The reducing agent was removed by dialysis against 100 volumes of borate phosphate buffer pH 7.6 for 12 h at 4° C. After this cycle of reduction/oxidation of the disulfide bridges, the proteins solution was loaded onto a G-50 Sephadex column and fractions were analysed for their protein contents by SDS-PAGE. A 10% tricine buffer was used and the gels were stained using silver staining.

Results and Discussion

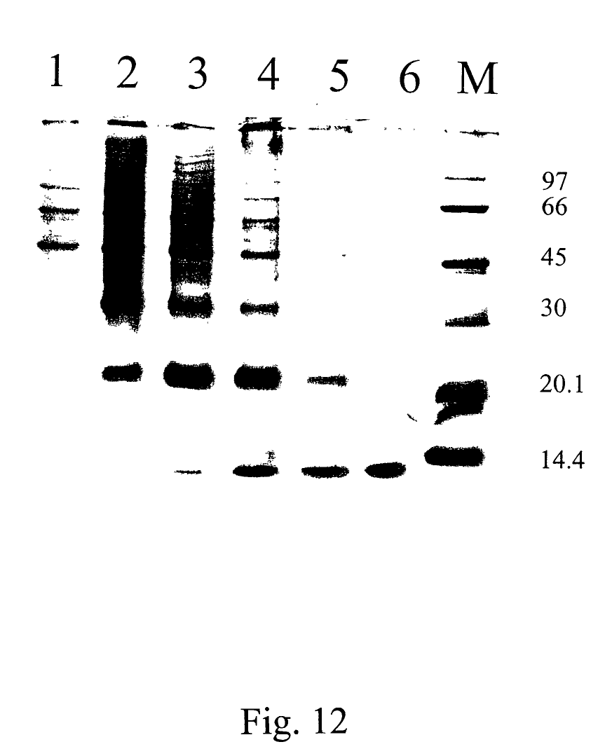

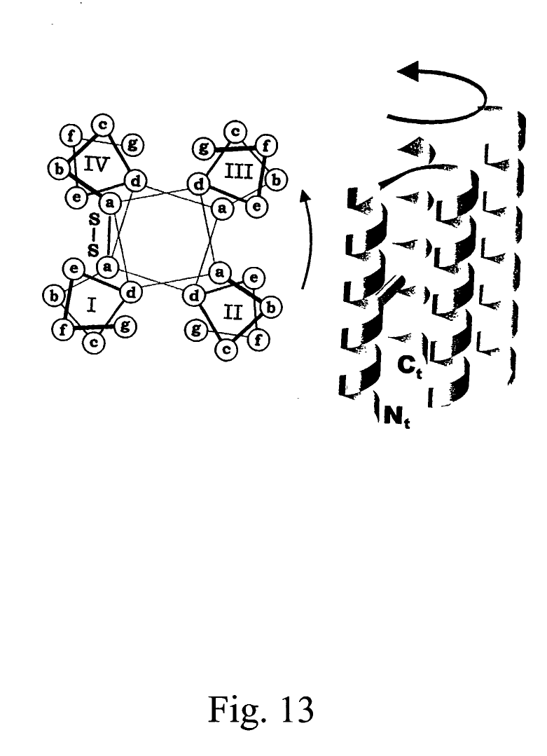

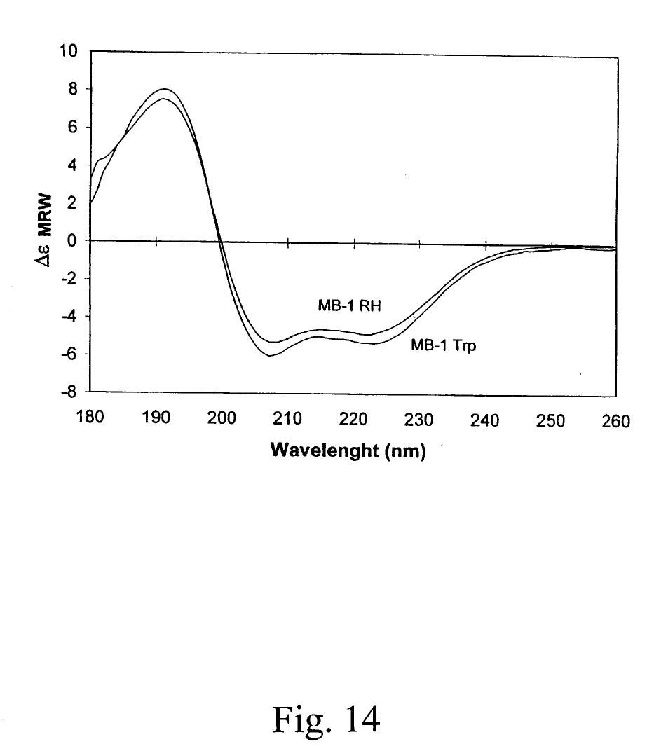

The present experiment illustrates a number of proteins intended to fold into α-helical proteins. One of the mutant we obtained, named MB-1Trp RH, is able to form intra-and inter-molecular disulfide bridges. Interestingly, the association of the mutant MB-1TrpRH is dependant on the way the oxidation of the bridges is carried out. The treatment described in Methods led to the formation of oligomers, with a rather even distribution of sizes in the range 11-125 kDa. The electrophoretic pattern of fractions collected from the size exclusion column is shown in FIG. 12. The fractions 5 and 6 contained mainly monomeric and dimeric MB-1TrpRH, migrating at 12 and 21 kDa respectively. The other lanes contained a number of oligomer proteins with the following estimated sizes: 12, 21, 35, 50, 60, 71, 79, 87 kDa, and other proteins of larger sizes (up to 125 kDa) that cannot be estimated with the gel system and protein markers used here. All these bands are in fact oligomers of MB-1TrpRH, a protein of 11.3 kDa. Circular dichroism (CD) measurements were performed and spectra typical of helical proteins were obtained (FIG. 13). Addition of DTT eliminated dimers and higher oligomers, which converted into monomers (FIG. 14).

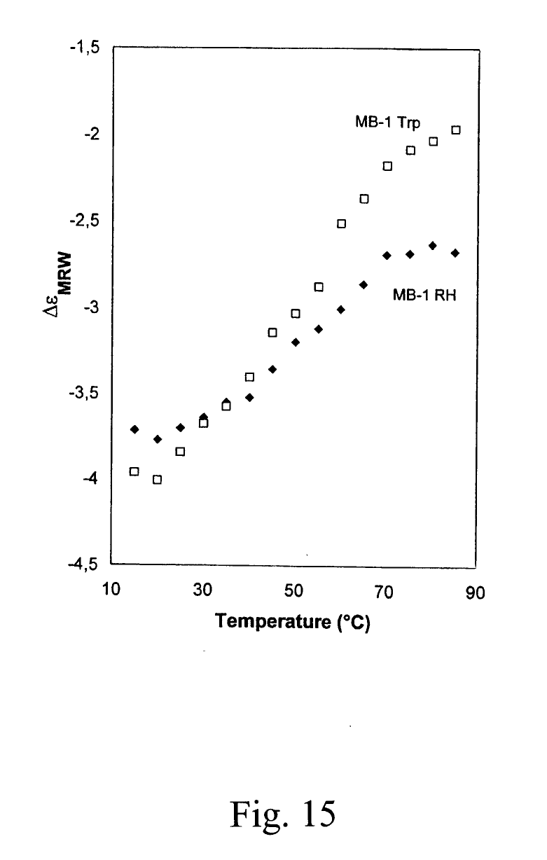

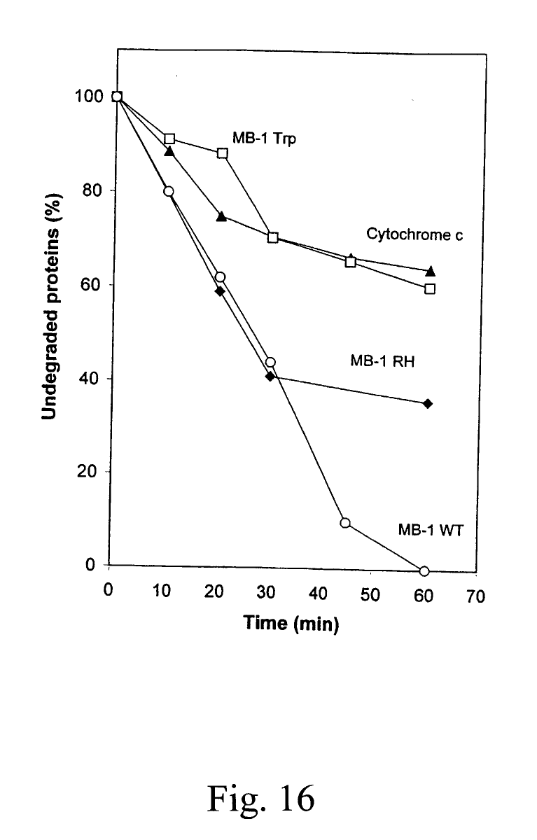

In the present experiment, MB-1TrpRH has 2 cysteines, therefore, one intra-molecular bridge may form in a monomer (FIG. 15). The protein has to form bridges with other monomers (i.e. the bridges cannot be intra-molecular) in order to form dimers or higher oligomers. Since those bridges involve Cys side chains that are expected to be in core positions in the monomers, it appears that formation of the ladder involve a drastic change in conformation for MB-1TrpRH subunits in dimer and higher oligomers. Interestingly, the proteins stay soluble, even after extensive cross-linking, which indicate that somehow, the core side chains are protected in the new cross-linked conformation. Degradation curves shown in FIG. 16 clearly demonstrate a gain in resistance to proteolytic attack.

Here the synthesis of various oligomers of a mutant proteins produced by a single bacterial clone was shown. After a cycle of reduction/oxidation of the disulfide bridges, the oligomer range in sizes from 11 kDa to approximately 125 kDa, and migrate as a ladder on a SDS-PAGE gel. Such a ladder is convenient in that it is made of the same proteins with size that are multiples of 11 kDa.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for improving supplying of at least one essential amino acid to a human or an animal, comprising administering to said human or animal at least one polypeptide having an amino acid sequence selected from the group consisting of SEQ ID NO: 1, SEQ ID NO: 2, SEQ ID NO: 3, SEQ ID NO: 4, and SEQ ID NO: 5, or a fragment thereof or at least one nucleic acid sequence encoding said polypeptide.

2. The method of claim 1, wherein said essential amino acid is a methionine, lysine, threonine, leucine, tryptophan, arginine, an analog or a derivative thereof.

3. The method of claim 1, wherein said animal is selected from the group consisting of a mammal, a bird, and a fish.

4. The method of claim 1, wherein said functional fragment comprises between 10 and 90 amino acids.

5. The method of claim 1, wherein said administration is an oral administration.

6. The method of claim 1, wherein said nucleic acid sequence is a DNA or a RNA sequence.

7. A composition comprising at least one polypeptide having an amino acid sequence selected from the group consisting of SEQ ID NO: 1, SEQ ID NO: 2, SEQ ID NO: 3, SEQ ID NO: 4 and SEQ ID NO: 5, or a functional fragment thereof, for endowing a human or an animal with at least one essential amino acid by oral administration.

8. The composition of claim 7, wherein said amino acid is a methionine, lysine, threonine, leucine, tryptophan, arginine, an analog or a derivative thereof.

9. The composition of claim 7, wherein said functional fragment comprises between 10 and 90 amino acids.

10. A polypeptide consisting of an amino acid sequence selected from the group consisting of SEQ ID NO: 1, SEQ ID NO: 2, SEQ ID NO: 3, SEQ ID NO: 4, and SEQ ID NO: 5 or a functional fragment thereof.

11. The polypeptide of claim 10, wherein said functional fragment comprises between 10 and 90 amino acids.

12. A nucleotide sequence encoding for a polypeptide as described in claim 10.

13. The nucleotide sequence of claim 12 being a DNA or a RNA sequence.

14. A nucleotide sequence selected from the group consisting of, SEQ ID NO: 6, SEQ ID NO: 7, SEQ ID NO: 8 SEQ ID NO: 9 and SEQ ID NO: 10 coding for a polypeptide as described in claim 9.

15. An expression vector comprising a nucleotide sequence as described in claim 12 or 14 designed for production of a recombinant polypeptide consisting of the amino acid sequence as set forth in SEQ ID NO: 1, SEQ ID NO: 2, SEQ ID NO: 3, SEQ ID NO: 4, and SEQ ID NO: 5, or a functional fragment thereof, said recombinant peptide being produced in a bacteria, yeast or an eukaryotic cell.

16. The expression vector of claim 15, wherein said functional fragment comprises between 10 and 90 amino acids.

17. A cell transformed with the expression vector as described in claim 15.
